(19) United States
(12) Patent Application Publication
(10) Pub. No.: US 2025/0361494 A1
(43) Pub. Date: Nov. 27, 2025

(54) ENGINEERED TN5 TRANSPOSASE COMPLEXES, METHODS OF PREPARING THE SAME, SYSTEMS AND METHODS OF PROFILING CO-OCCURRING CHROMATIN FEATURE IN A CELL

(71) Applicant: The Hong Kong University of Science and Technology, Hong Kong (CN)

(72) Inventors: Ming Fung CHEUNG, Hong Kong (CN); Chi Yeu LEUNG, Hong Kong (CN)

(21) Appl. No.: 19/196,786

(22) Filed: May 2, 2025

Related U.S. Application Data
(60) Provisional application No. 63/650,418, filed on May 22, 2024.

(51) Int. Cl. C12N 9/12; C07K 16/18; C12N 9/50; C12Q 1/6874

(52) U.S. Cl. CPC C12N 9/1241; C07K 16/18; C12N 9/50; C12Q 1/6874; C07K 2319/30; C12Y 304/2207

(57) ABSTRACT

In some embodiments, provided is an engineered Tn5 transposase complex, comprising: at least one Tn5 transposase, comprising a poly-glycine sequence; and at least one antibody that is covalently linked to the at least one Tn5 transposase by a sortase, wherein the at least one antibody that is specific to a target site of a chromatin sequence. Other example embodiments are described herein. In certain embodiments, provided engineered Tn5 transposase complexes, systems, methods and kits provide outstanding performance in identifying chromatin features especially co-occurrence of chromatin features in samples and enable identifying multiple chromatin features at single cell resolution.

### CROSS-REFERENCE TO RELATED APPLICATION

This application claims benefit under 35 U.S.C. § 119(e) of U.S. Provisional Application having Ser. No. 63/650,418 filed May 22, 2025, the entire contents of which is/are hereby incorporated by reference herein.

COPYRIGHT NOTICE: A portion of the disclosure of this patent document contains material, which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### REFERENCE TO SEQUENCE LISTING

This application contains one or more sequence listings in computer readable form, which are incorporated herein by reference in their entireties. This application contains a sequence listing which has been submitted electronically in ST.26 (xml) format and is hereby incorporated by reference in its entirety. Said ST.26 copy, created on Mar. 27, 2025, is named “H0010000006DRF.xml” and is 3 kilobytes in size.

### FIELD OF INVENTION

The present invention generally relates to the field of epigenomic profiling. More specifically the present invention relates to providing a low-input and single-cell resolution epigenomic profiling method for co-occurring chromatin features.

### BACKGROUND OF INVENTION

Post-translation modifications (PTMs) of histone proteins are essential to transcription regulation and chromatin organization 1. Different types of histones PTMs are enriched at specific genomic contexts. The combination of various modifications confers distinct transcriptional states. Moreover, the PTM signatures at sequences are used to define their functionality1-. For example, histone H3 lysine 4 tri-methylation (H3K4me3) is found at active gene promoter elements, whereas H3 lysine 27 tri-methylation (H3K27me3) is enriched at repressed elements.

Interestingly, analysis of differentiating embryonic stem cells revealed a phenomenon termed as bivalency. In these cells, poised developmental gene promoters are marked by both H3K4me3 and H3K27me3. Upon differentiation into committed cell lineages, these elements lose either the active or repressive modifications, presumably to achieve rapid gene activation or silencing during early embryonic development.

In subsequent studies, bivalency of PTMs was also found in a number of other cell types. However, traditional methods to define histone PTMs, such as chromatin immunoprecipitation sequencing (ChIP-seq), cannot effectively determine the co-existence of both modifications on the same chromatin segment. This is because ChIP-seq utilizes antibodies to recognize histones associated with a particular PTM, which are then be “pulled-down”. The results represent the averaged picture of a heterogenous population of millions of cells.

To address this problem, techniques like sequential ChIP (ChIP-re-ChIP) and co-ChIP, were developed specifically to study the co-occurrence of histone PTMs. ChIP-re-ChIP implements successive rounds of chromatin immunoprecipitations using different antibodies, while Co-ChIP uses combinatorial indexing of immunoprecipitated chromatin. As these ChIP-based methods involve repeated washing of pulled-down chromatin, large amounts of starting input materials are required. These protocols are also laborious and dependent on highly efficient antibodies, which traditionally vary greatly between lots.

Recently, F Cleavage Under Targets and Tagmentation (CUT&Tag) was developed to improve on existing approaches. Protein A (pA)-Tn5 transposase fusion is used to associate antibodies to direct the enzyme to genomic sequences marked with PTM of interest, which then introduces sequencing adapters to the locus. Subsequently, next generation sequencing (NGS) libraries are generated via PCR targeting the adapters. CUT&Tag can be performed on low cell numbers, and the protocol can be completed within one day. However, while this represents a step forward, the weak attachment of antibodies to the fusion proteins still harbors issues of low efficiency and variability between antibodies.

### SUMMARY OF INVENTION

Disclosed herein are novel engineered Tn5 transposase complexes, methods of preparing the complexes, kits thereof and uses thereof, systems and methods of profiling co-occurring chromatin feature(s) in a cell, methods of using the complexes, and intermediates used in preparing the complexes. In some examples, provided systems or methods of profiling co-occurring chromatin feature(s) is called “ReACT-seq”.

In some embodiments, provided is an engineered Tn5 transposase complex, including: at least one Tn5 transposase, including a poly-glycine sequence; and at least one antibody that is covalently linked to the at least one Tn5 transposase by a sortase, wherein the at least one antibody is specific to a target site of a chromatin sequence.

In some embodiments, provided is a method of preparing recombinant Tn5 transposase complex, including the steps of: (a) preparing at least one Tn5 transposase including poly-glycine sequence; and (b) covalently linking the at least one Tn5 transposase including poly-glycine sequence with at least one antibody including a LPXTG-motif tag by a sortase, wherein the at least one antibody that is specific to a target site of a chromatin sequence.

There are many advantages of the invention. In certain embodiments, provided complexes, methods, systems have outstanding performance in identifying chromatin features especially co-occurrence of chromatin features in samples. In certain embodiments, provided complexes, methods, systems have high efficiency and solved the problems of variability between antibodies. In certain embodiments, the recombinant antibodies are generated in vitro, consistency between lots or batches is maintained. In certain embodiments, provided complexes, methods, systems can be directly and strongly conjugated to a diverse collection of recombinant antibodies, enabling to be scaled to a single-cell level or resolution.

### BRIEF DESCRIPTION OF FIGURES

For a better understanding of the various embodiments described herein and to show more clearly how these various embodiments may be carried into effect, reference will be made, by way of example, to the accompanying drawings, which show at least one example embodiment, and which are now described.

FIG. 1 is a schematic diagram illustrating the system of epigenomics mapping of co-occurring chromatin features. The recombinant antibodies are covalently bonded to the engineered Tn5 through sortase, which is further equipped with adapter for consequent PCR amplification once the system bonds to the target sites on the chromatin sequence.

FIG. 2 is a schematic diagram illustrating the process to generate adapter-Tn5-antibody complex by the action of sortase on recombinant antibody and the engineered Tn5.

FIG. 3 is a schematic diagram illustrating the application of ReACT-seq to identify co-occurring chromatin features on the same DNA molecule.

FIG. 4 is a schematic diagram illustrating the application of ReACT-seq to identify multiple chromatin features at single cell resolution.

FIGS. 5A-5D are schematic diagrams illustrating complexes and systems of epigenomics mapping of co-occurring chromatin features. The recombinant antibodies are covalently bonded to the engineered Tn5 through sortase, which is further equipped with adapter for consequent PCR amplification once the system bonds to the target sites on the chromatin sequence.

FIG. 6 is a schematic diagram illustrating the process to generate adapter-Tn5-antibody complex by the action of sortase on recombinant antibody and the engineered Tn5. Sortase and recombinant antibody is added to the engineered Tn5, where sortase then mediates linkage between Tn5 and the recombinant antibody. The resulting engineered Tn5-antibody complex is recovered as a product of this reaction.

FIG. 7 is a schematic diagram illustrating the application of ReACT-seq to identify co-occurring chromatin features on the same DNA molecule. The engineered Tn5-antibody complex is introduced, enriching the complex to bind to their specific target site on a chromatin. The complex fragments and adds adapter to the chromatin, forming fragments with different adapter at each end. The fragments are purified and sequencing platform specific adapters are added through PCR to form the final product for NGS sequencing.

FIG. 8 is a schematic diagrams illustrating the application of ReACT-seq to identify multiple chromatin features at single cell resolution. The engineered Tn5-antibody complex is introduced, enriching the complex to bind to their specific target site on a chromatin. The complex fragments and adds adapter to the chromatin, forming fragments with different adapter at each end. Cell barcode and sequencing adapter is added to the fragments using microfluidic device. A second sequencing adapter is added through tagmentation. The product is finally purified and amplified with PCR for NGS sequencing.

FIG. 9 showed genome browser tracks illustrating NGS library generated with ReACT-seq protocol, displaying a signal profile similar or comparable to NGS library generated with CUT&Tag protocol.

FIG. 10. showed genome browser tracks illustrating ReACT-seq identifying co-occurrence of H3K4me3 and H3K27me3.

FIG. 11 showed genome browser tracks illustrating ReACT-seq identifying regions comparable to re-ChIP.

FIG. 12. is a Venn diagram showing genome wide overlapping of peaks called from ReACT-seq and reChIP.

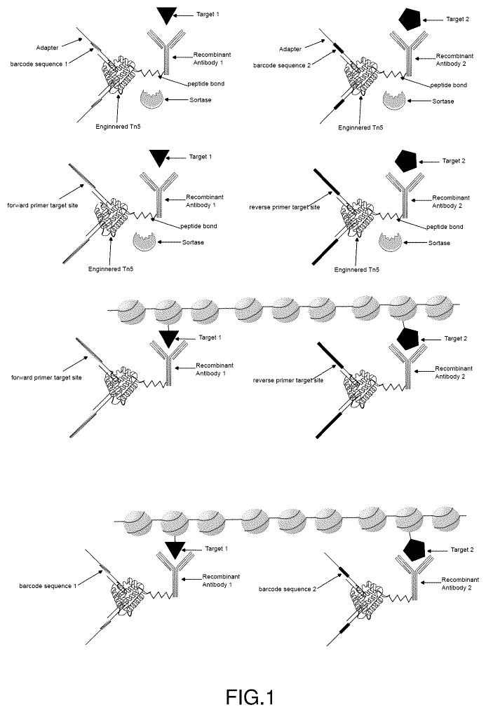

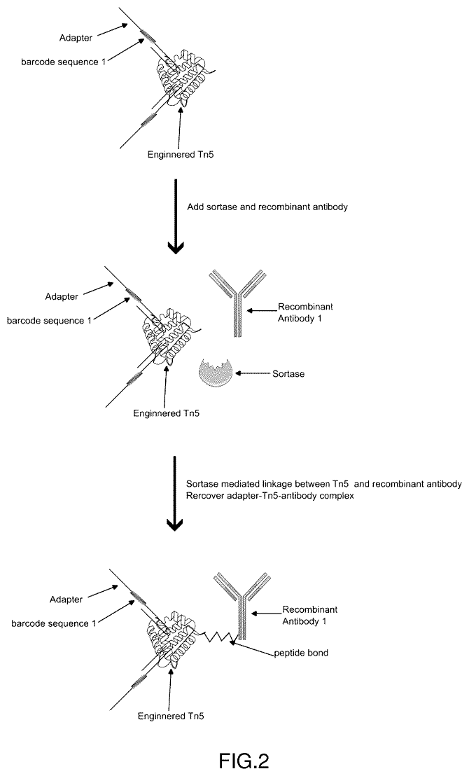

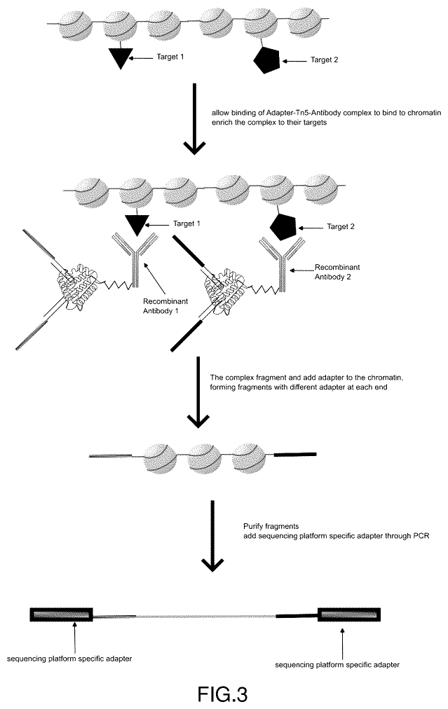

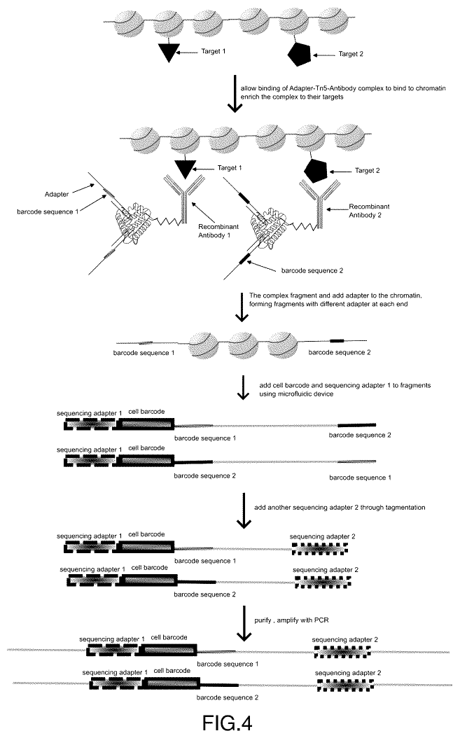

### DETAILED DESCRIPTION

As used herein and in the claims, the terms “comprising” (or any related form such as “comprise” and “comprises”), “including” (or any related forms such as “include” or “includes”), “containing” (or any related forms such as “contain” or “contains”), means including the following elements but not excluding others. It shall be understood that for every embodiment in which the term “comprising” (or any related form such as “comprise” and “comprises”), “including” (or any related forms such as “include” or “includes”), or “containing” (or any related forms such as “contain” or “contains”) is used, this disclosure/application also includes alternate embodiments where the term “comprising”, “including,” or “containing,” is replaced with “consisting essentially of” or “consisting of”. These alternate embodiments that use “consisting of” or “consisting essentially of” are understood to be narrower embodiments of the “comprising”, “including,” or “containing,” embodiments.

For example, alternate embodiments of “a composition comprising A, B, and C” would be “a composition consisting of A, B, and C” and “a composition consisting essentially of A, B, and C.” Even if the latter two embodiments are not explicitly written out, this disclosure/application includes those embodiments. Furthermore, it shall be understood that the scopes of the three embodiments listed above are different.

For the sake of clarity, “comprising”, including, and “containing”, and any related forms are open-ended terms which allows for additional elements or features beyond the named essential elements, whereas “consisting of” is a closed end term that is limited to the elements recited in the claim and excludes any element, step, or ingredient not specified in the claim.

For the sake of clarity, “characterized by” or “characterized in” (together with their related forms as described above), does not limit or change the nature of whether the list of terms following it are open or closed. For example, in a claim directed towards “a composition comprising A, B, C, and characterized in D, E, and F”, the elements D, E, and F are still open-ended terms and the claim is meant to include other elements due to the use of the word “comprising” earlier in the claim.

“Consisting essentially of” limits the scope of a claim to the specified materials, components, or steps that do not materially affect the essential characteristic(s) of the claimed invention. In some embodiments, the essential characteristics are the basic and novel characteristic(s) of the claimed invention.

As used herein, the singular forms “a”, “an” and “the” are intended to include the plural forms as well, unless the context clearly indicates otherwise. Where a range is referred in the specification, the range is understood to include each discrete point within the range. For example, 1-7 means 1, 2, 3, 4, 5, 6, and 7.

As used herein, the term “about” is understood as within a range of normal tolerance in the art and not more than ±10% of a stated value. By way of example only, about 50 means from 45 to 55 including all values in between. As used herein, the phrase “about” a specific value also includes the specific value, for example, about 50 includes 50.

As used herein, the term “Transposon 5 transposase”, “Tn5 transposase” or “Tn5” is an enzyme that cuts and inserts DNA fragments (such as transposons) into new locations in the genome, which may be used in genetic engineering and next-generation sequencing library preparation such as tagmentation.

As used herein, the term “Tn5 transposase complex” or “complex” is a biomolecular assembly that contains at least one Tn5 transposase and one or more other elements, such as antibody and/or adapter.

As used herein, the term “sortase” refers to an enzyme that links or connects a protein (such as Tn5 Transposase) to another one (e.g., an antibody) by forming covalent bonds therebetween. In some examples, sortase cleaves a specific motif, such as LPXTG motif, in a target protein.

As used herein, the term “poly-glycine sequence” refers to a sequence or fragment containing more than one (multiple) glycine (Gly, G) amino acids repeated consecutively. In some examples, the poly-glycine sequence is covalently linked to a Tn 5 transposase. Examples of poly-glycine sequence such as polyglycine-5 (GGGGG) or polyglycine-10 (GGGGGGGGGG).

As used herein, the term “antibody” is a protein that recognizes and neutralize an antigen. In some examples, an antibody is specific to a target site or chromatin feature on the chromatin.

As used herein, the term “chromatin” is a complex of DNA and protein (such as histone) found in eukaryotic cells. As used herein, the term “chromatin feature” refers to the structural or functional characteristics of chromatin. As used herein, the term “co- occurring chromatin feature” refers to two or more (multiple) chromatin features appear together in the same region of a chromatin.

As used herein, the term “adapter” or “sequencing adapter” is a synthetic oligonucleotide that contains sequences that can be recognized by sequencing instruments for sequencing (e.g., next-generation sequencing.)

As used herein, the term “barcode sequence” is a unique nucleotide (e.g., DNA) sequence (e.g., 6-12 nucleotides) added to the adapter such as to identify a target molecule.

As used herein, the term “tagmentation” refers to a process in next-generation sequencing (NGS) library preparation where a transposase simultaneously fragments DNA (e.g., on chromatin) and inserts adapter(s) in a single step.

As used herein, the term “LPXTG-motif” is a sequence that can be recognized/cleaved by a sortase to covalently link a protein (e.g., an antibody) to the other protein.

Although the description referred to particular embodiments, the disclosure should not be construed as limited to the embodiments set forth herein.

### Numbered Embodiments

### Set 1

Embodiment 1: A low-input, single-cell resolution epigenomic profiling system for co-occurring chromatin features, comprising:
one or more engineered Tn5 transposase comprising an added poly-glycine sequence and adapter sequences;
one or more recombinant antibodies carrying a LPXTG-motif tag, wherein X is any single amino acid and wherein the recombinant antibodies comprise specific binding sites for tagging specific sequences on the chromatin fragment; and
a sortase enzyme to covalently link the Tn5 transposase and recombinant antibodies;
wherein the Tn5 transposase-recombinant antibodies complex tag the fragments corresponding to co-occurring chromatin features for subsequent PCR amplification for downstream library preparation and fragment deciphering.

Embodiment 2: A method to produce next generation sequencing libraries for defining co-occurring chromatin features, using the profiling system of claim

### Set 2

Embodiment 1. An engineered Tn5 transposase complex, comprising: at least one Tn5 transposase, comprising a poly-glycine sequence; and at least one antibody that is covalently linked to the at least one Tn5 transposase by a sortase, wherein the at least one antibody is specific to a target site of a chromatin sequence.

Embodiment 2. The engineered Tn5 transposase complex of embodiment 1, further comprises at least one adapter.

Embodiment 3. The engineered Tn5 transposase complex of any one of the preceding embodiments, wherein the at least one adaptor comprises at least one barcode sequence.

Embodiment 4. A plurality of engineered Tn5 transposase complexes as described in any one of the preceding embodiments, wherein each complex comprises a different antibody that is specific to a different target site of the chromatin sequence.

Embodiment 5. The plurality of complex of any one of the preceding embodiments, wherein each complex further comprises at least one different adapter comprising at least one different barcode sequence.

Embodiment 6. A system of profiling co-occurring chromatin features, comprising a plurality of engineered Tn5 transposase complexes as described in any one of the preceding embodiments, wherein each complex comprises a different antibody comprising a different specific binding site for tagging a different specific sequence on the at least one target chromatin.

Embodiment 7. The system of embodiment 6, wherein each complex further comprises at least one different adaptor comprising at least one different barcode sequence.

Embodiment 8. The system of embodiment 6 or 7, comprising: a first engineered Tn5 transposase complex as described in any one of the preceding embodiments, wherein the first complex comprises a first antibody comprising a first specific binding site for tagging a first specific sequence on a target chromatin; and a second engineered Tn5 transposase complex as described in any one of the preceding embodiments, wherein the second complex comprises a second antibody comprising a second specific binding site for tagging a second specific sequence on the target chromatin, wherein the first engineered Tn5 transposase complex further comprises at least one first adapter, and the second engineered Tn5 transposase complex further comprises at least one second adapter, and wherein the first adapter comprises a first barcode sequence and the second adapter comprises a second barcode sequence that is different from the first barcode sequence.

Embodiment 9. A method of profiling co-occurring chromatin features using a plurality of complexes as described in any one of embodiments 1 to 5 or the system of any one of embodiments 6 to 8, comprising the steps of: (i) reacting the plurality of complexes with a sample such that the plurality of complexes bind to the any target site of the chromatin sequence of the sample to obtain a chromatin fragment, (ii) performing tagmentation on the chromatin fragment in situ; and (iii) preparing sequencing library for sequencing.

Embodiment 10. The method of embodiment 9, wherein each complex further comprises at least one different adaptor.

Embodiment 11. The method of embodiment 9 or 10, wherein the at least one different adaptor comprises at least one different barcode sequence.

Embodiment 12. The method of any one of embodiments 9 to 11, prior to the step (iii), further comprises the steps of: purifying the chromatin fragment and adding sequencing platform specific adapter at each end of the chromatin fragment by PCR.

Embodiment 13. The method of any one of embodiments 9 to 12, wherein at least one or more of the steps are performed in a microfluidic device.

Embodiment 14. A method of preparing recombinant Tn5 transposase complex, comprising the steps of: (a) preparing at least one Tn5 transposase comprising poly-glycine sequence; and (b) covalently linking the at least one Tn5 transposase comprising poly-glycine sequence with at least one antibody comprising a LPXTG-motif tag by a sortase, wherein the at least one antibody that is specific to a target site of a chromatin sequence.

Embodiment 15. The method of embodiment 14, wherein the step (a) further comprises the steps of: preparing the at least one Tn5 transposase comprising poly-glycine sequence by reacting TEV-Gly5-Tn5 with a TEV-protease.

Embodiment 16. The method of embodiment 15, the TEV-Gly5-Tn5 is prepared by replacing 3XFlag-pA portion of a 3XFlag-pA-Tn5-FI plasmid by PCR.

Embodiment 17. The method of any one of embodiments 14 to 16, further comprising a step of: prior to the (b), purifying the recombinant Tn5 transposase complex.

Embodiment 18. The method of any one of embodiments 14 to 17, further comprising a step of: reacting the recombinant Tn5 transposase complex with at least one adapters to form adaptor loaded Tn5 transposase complex.

Embodiment 19. The method of embodiment 18, wherein the at least one adaptor comprises at least one barcode sequence.

Embodiment 20. A kit of profiling at least one co-occurring chromatin feature in a cell, comprising: a. at least one Tn5 transposase comprising a poly-glycine sequence; b. at least one antibody comprising a LPXTG-motif tag, wherein X is any single amino acid and wherein the at least one antibody comprises a specific binding site for tagging a specific sequence on a chromatin fragment; c. at least one adapter sequence; and d. a sortase enzyme that is configured to covalently linking the Tn5 transposase and the at least one antibody.

Embodiment 21. The kit of embodiment 20, comprises a first antibody comprising a first specific binding site for tagging a first specific sequence on a chromatin fragment; a second antibody comprising a second specific binding site for tagging a second specific sequence on a chromatin fragment; a first adapter sequence comprising a first barcode sequence; and a second adapter sequence comprising a first barcode sequence.

### EXAMPLES

Provided herein are examples that describe in more detail certain embodiments of the present disclosure. The examples provided herein are merely for illustrative purposes and are not meant to limit the scope of the invention in any way. All references given below and elsewhere in the present application are hereby included by reference.

### Example 1: Low-Input and Single-Cell Epigenomic Profiling Method for Co-Occurring Chromatin Features

### Detailed Description:

In the following description, methods and modification procedures of aptamers as target detectors are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

In recent years, the Tn5 transposase enzyme has been utilized in a variety of important genomics approaches, such as the Nextera library preparation, ATAC-seq, and CUT&Tag. In a process coined as tagmentation, hyperactive Tn5 can randomly cut DNA or chromatin and insert specific sequences to the cut site 9. Therefore, researchers have exploited this property to use transposases loaded with designed DNA sequences called mosaic end double strand oligonucleotides (MEDS). For NGS, the MEDS allow for introduction of sequencing adapters into random sites of the template. Utilizing distinct MEDS can increase the complexity of the application. For example, MEDS-A and MEDS-B, which harbor different adapter sequences can be loaded at equimolar concentrations to Tn5 enzymes. When added to a DNA sample, the transposase would fragment the sample and transfer a strand of a MEDS to the template. Given random transposition events, on average, 50% of fragmented products will carry both MEDS-A and MEDS-B adapters, 25% will have only MEDS-A, and 25% will have only MEDS-B. Those that contain both MEDS-A and MEDS-B adapters can be preferentially amplified by PCR. This process is used for efficient and quick generation of sequencing libraries for the Illumina and BGI massive parallel sequencing platforms.

In 2019, the Henikoff lab invented CUT&Tag, a method that utilizes a protein A (pA) and Tn5 fusion protein to investigate the enrichment patterns of histone PTMs and chromatin components 8. Specific antibodies are associated to the pA-Tn5 protein, which mediates targeted tagmentation. This represented a significant improvement over traditional ChIP-seq assays, reducing the input from millions of cells down to thousands or even hundreds.

Also, it has substantially less background signal, which translates to much lower required sequencing depth, decreasing the minimum sequencing reads down to about 1/10 of ChIP-seq. This technique was reported to scale down to the single-cell level for H3K27me3 in cultured immortalized cancer cells 8.

However, given the relatively weak association between pA and antibodies, allele dropouts or false negatives remain a potential issue. As commercially available monoclonal antibodies are notoriously variable in specificity and affinity, we and others have noted that while CUT&Tag works well for some antibodies, it fails with others. These issues are exacerbated when using low cell numbers. To circumvent these problems, the ReACT-seq is developed, which uses an engineered Tn5 enzyme, which can be directly and strongly conjugated to a diverse collection of recombinant antibodies.

This application aims to demonstrate a method that enables low-input, simultaneous, genome-wide profiling of multiple post-translation modifications (PTMs) of histone proteins. In this method, engineered Tn5 transposase is directly conjugated to recombinant antibodies, forming a tethered complex. The specificity of the antibody would direct the enzymatic component to the regions of the genome enriched with the histone PTMs targeted by the conjugated antibody. By loading distinct barcoded sequencing adapters to different tethered complexes, we can elucidate the co-occurrence of multiple histone PTMs. Next-generation sequencing libraries are then generated by PCR amplification targeting a specific combination of adapters, thereby allowing genome-wide mapping of two or more histone PTMs at once. In combination with droplet-based techniques, this method can be scaled to a single-cell level.

### Summary of Invention

In ReACT-seq, a novel Tn5 transposase is produced with an added poly-glycine sequence. In addition, recombinant antibodies carrying a tag with the LPXTG motif are utilized (X can be any amino acid). When treated with the Sortase enzyme, the motif on the antibody will be cleaved between the threonine and glycine residues and the terminal glycine is then replaced with any poly-glycine sequences. As a result, the two components are covalently linked together. The antibody is tightly bound to the Tn5, which can then be used for guided tagmentation on chromatin in situ. Given that the recombinant antibodies are generated in vitro, consistency between lots or batches is maintained.

When engineering and purifying Tn5, we customize the carried adapter sequences. To simultaneously detect the binding of 2 different antibody-Tn5 complexes to a single chromatin fragment, we load each complex with distinct oligonucleotides. For example, Tn5 conjugated to antibodies 1 and 2 are loaded with adapter 1 and adapter 2, respectively. When introduced to permeabilized nuclei or cells, antibody tethered Tn5 will bind and tag their targets. Fragments containing both adapter 1 and adapter 2 would indicate the co-occurrence of both modifications of interest. Whereas regions enriched with either one of the epitopes of antibodies 1 or 2 will produce DNA fragments with the same adapters on both ends.

In the downstream library preparation, the fragments with both adapter 1 and adapter 2 are preferentially PCR amplified. Therefore, ReACT-seq can decipher the co-occurrence of multiple-chromatin features on individual fragments.

### Example 2: Engineered Tn5 Transposase Complexes, Methods of Preparing the Same, and Systems and Methods of Profiling Co-Occurring Chromatin Features

### 2.1 Engineered Tn5 Transposase Complexes

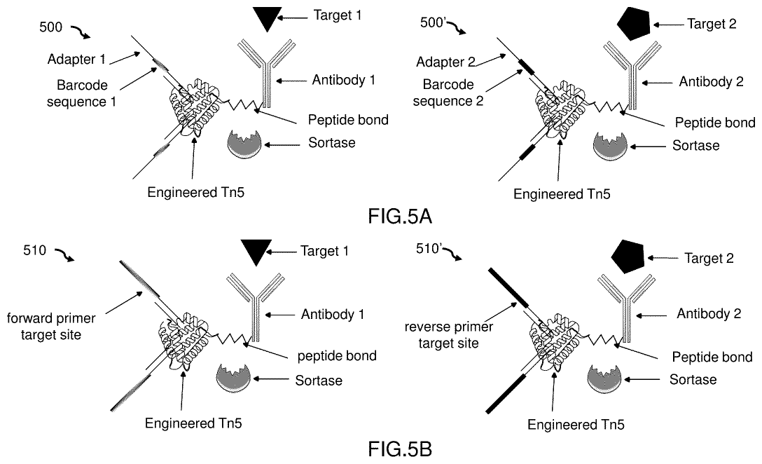

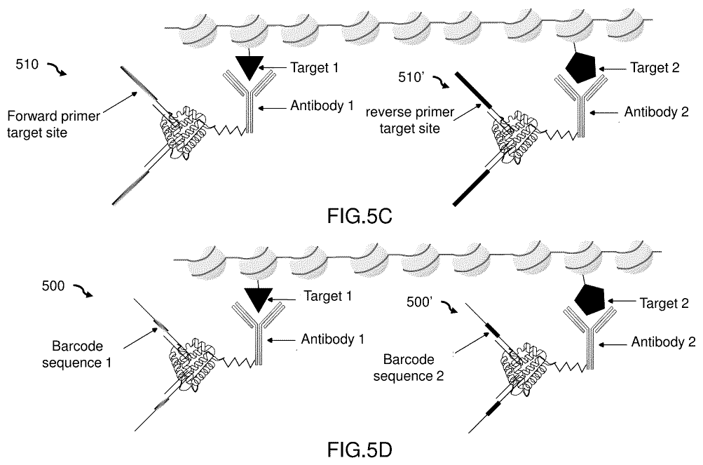

Now referring to FIGS. 5A, showing two example engineered Tn5 transposase complexes 500 and 500′ (collectively, 500). The engineered Tn5 transposase complex 100 generally contains at least one Tn5 transposase that contains a poly-glycine sequence, and at least one antibody that is covalently linked to the at least one Tn5 transposase by a sortase, wherein the at least one antibody is specific to a target, such as a target site of a chromatin sequence. In this example, the Tn5 transposase complex further contains or equipped with at least one adapter for consequent PCR amplification once the complex or system bonds to the target sites on the chromatin sequence. In this example, engineered Tn5 transposase complex 500 contains an engineered Tn5 transposase containing a poly-glycine sequence, and an antibody 1 that is covalently linked to the Tn5 transposase at the poly-glycine sequence by a sortase with a peptide bond, wherein the antibody 1 (which is different from antibody 2) is specific to the target 1 (which is different from target 2). Engineered Tn5 transposase complex 500 also contains a pair of adapters 1. The engineered Tn5 transposase complex 500′ contains an engineered Tn5 transposase containing a poly-glycine sequence, and antibody 2 that is covalently linked to the Tn5 transposase by a sortase, wherein the antibody 2 is specific to the target 2. Engineered Tn5 transposase complex 500′ also contains a pair of adapters 2. In this example, each adapter 1 further contains a unique barcode sequence 1 and each adapter 2 contains a unique barcode sequence 2 which is different from barcode sequence 1.

Now referring to FIG. 5B, showing another two example engineered Tn5 transposase complexes 510 and 510′ (collectively, 510). The engineered Tn5 transposase complexes 510 are structurally similar to engineered Tn5 transposase complexes 500, except the pair of adapters 1 and 2 contain forward primer target site and reverse primer target site, respectively.

Now referring to FIG. 5C, showing the target 1 and the target 2 are present on a chromatin sequence. The two example engineered Tn5 transposase complexes 510 and 510′ (which contains the antibodies 1 and 2 tethered Tn5 transposase and forward and reverse primer target sites) bind and tag to the respective targets 1 and 2 on the chromatin sequence.

Now referring to FIG. 5D, showing the target 1 and the target 2 are present on a chromatin sequence. The two example engineered Tn5 transposase complexes 500 and 500′ (whichcontains the antibodies 1 and 2 tethered Tn5 transposase and barcode sequences 1 and 2) bind and tag to the respective targets 1 and 2 on the chromatin sequence.

In other examples, one example engineered Tn5 transposase complex is provided to identify a chromatin feature. In some other examples, more than two (e.g., 3, 4, 5, 6, 7, 8, 9 or 10 or more) example engineered Tn5 transposase complexes are provided to identify multiple chromatin features.

### 2.2 Methods of Preparing Tn5 Transposase Complexes

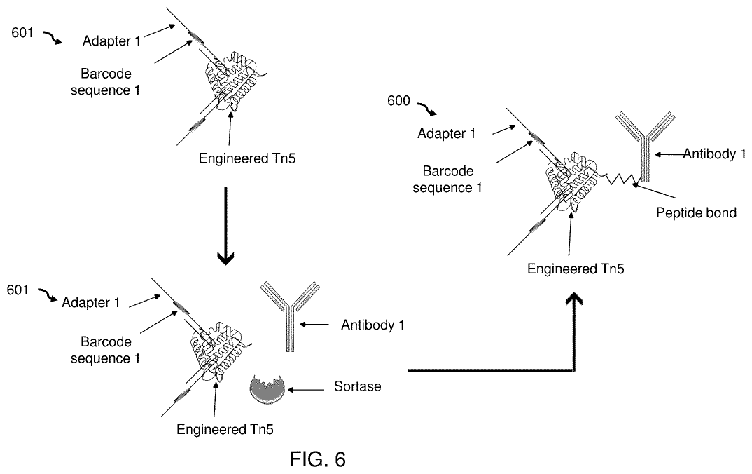

Now referring to FIG. 6, showing the process of preparing an example Tn5 transposase complex. An adapter-Tn5-antibody complex by the action of sortase on recombinant antibody 1 and the engineered Tn5 is prepared. Firstly, an engineered Tn5 601 which contains a pair of adapter 1 (which contains barcode sequence 1) and poly-glycine sequence is prepared to form an adapter-Tn5 complex. Then, the adapter-Tn5 complex is incubated with an antibody 1 (which contains a tag with the LPXTG motif, wherein X can be any amino acid motif) and a sortase. The antibody 1 is cleaved between the threonine and glycine residues at the LPXTG motif and the terminal glycine is then replaced with the poly-glycine sequences at the engineered Tn5 transposase by the sortase enzyme, to form the adapter-Tn5-antibody complex 600.

### 2.3 Systems and Methods of Profiling Co-Occurring Chromatin Features

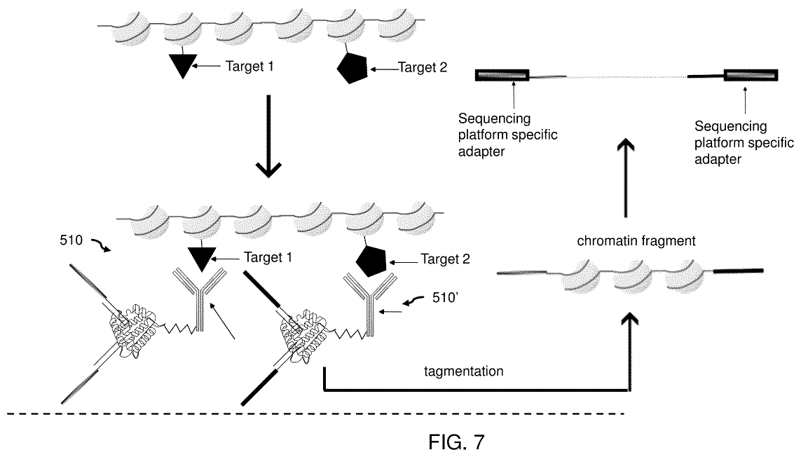

Now referring to FIG. 7, showing example systems and methods of profiling co-occurring chromatin features (namely, ReACT-seq). In this example, a sample containing a fragment of chromatin containing two different targets (target 1 and target 2) (i.e., having co-occurring chromatin features on the same DNA molecule) is prepared from nuclei or cells of interest. The example engineered Tn5 transposase complexes (such as complexes 510 and 510′) are prepared and incubated with the sample, such that the antibody 1 and antibody 2 of the complexes 510 and 510′ bind to the target 1 and target 2, respectively, so as to enrich the complexes to their respective targets. As such, a complex chromatin fragment flanked by different adapters (the adapters 1 and 2) at each end is formed by tagmentation. The Tn5 transposase cuts the chromatin fragment and ligates the adapters to both ends of the fragment. If the sample does not contain co-occurring chromatin features (e.g., contain only one or none of the targets), such fragment flanked by different adapters at each end may not be formed. Optionally and additionally, the chromatin fragment is further purified and sequencing platform specific adapters are introduced or added to the ends by PCR, such that NGS library and sequencing can be performed. As such, co-occurring chromatin features on the same DNA molecule can be identified by ReACT-seq.

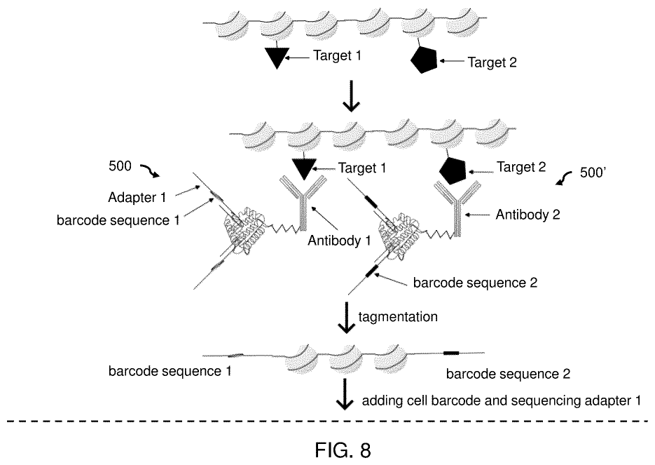

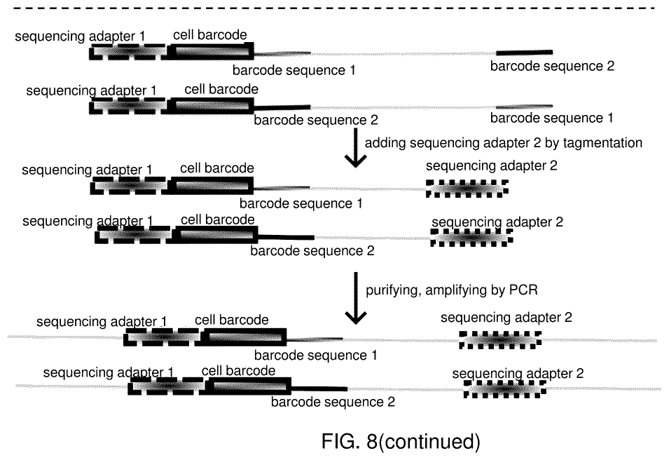

Now referring to FIG. 8, showing example systems and methods of profiling multiple chromatin features (namely, ReACT-seq) at a single cell resolution. In this example, a sample containing a fragment of chromatin containing two different targets (target 1 and target 2) (i.e., having co-occurring chromatin features on the same DNA molecule) is prepared from a single nucleus or cell of interest. The example engineered Tn5 transposase complexes (such as complexes 500 and 500′) are prepared and incubated with the sample, such that the antibody 1 and antibody 2 of the complexes 500 and 500′ bind to the target 1 and target 2, respectively, so as to enrich the complexes to their respective targets. As such, a complex chromatin fragment flanked by different barcode sequences (the barcode sequences 1 and 2) at each end is formed by tagmentation. The Tn5 transposase cuts the chromatin fragment and ligates the adapters to both ends of the fragment. If the sample does not contain co-occurring chromatin features (e.g., contain only one or none of the targets), such fragment flanked by different adapters at each end may not be formed. Optionally and additionally, the chromatin fragment is further purified, and a cell barcode and sequencing platform specific adapter (sequencing adapter 1) are introduced or added to one of the ends by PCR. Then, another sequencing adapter 2 is introduced or added to the other end by tagmentation. After purifying and amplifying by PCR, products containing 2 sequencing adapters at both ends with different barcode sequences are obtained, such that sequencing such as NGS can be performed. As such, co-occurring chromatin features in a single cell can be identified by ReACTseq.

In some examples, at least one or more of these steps are performed using a microfluidic device.

### Example 3: Preparation of Tn5 Transposase Comprising Poly-Glycine Sequence

### 3.1 Molecular Cloning of TEV-Gly5-Tn5 Plasmid

1. Using a commercially available 3XFlag-pA-Tn5-FI plasmid (Addgene plasmid #124601), the 3XFlag-pA portion is replaced with TEV-Gly5 (peptide sequence containing a Tobacco Etch Virus (TEV) protease cleavage site followed by five glycine residues), by PCR using primers EPILAB0255 and EPILAB0256. The primer information are summarized as below.

TABLE 1
Primer sequence information

| Primer | | |
|---|---|---|
| Name | Sequence | SEQ ID No. |
| EPILAB0255 | ggtggtggcggcggtgatgacgataaagaattcg | 1 |
| EPILAB0256 | ttggaaatacaggttctccatggtatatctccttct | 2 |

2. Blunt-end ligate the PCR product and transform to NEB stable Competent E. coli (NEB C3040). TEV-Gly5-Tn5 plasmid was obtained.

3. Obtain the plasmid by miniprep kit (Invitrogen, K210010) according to manufacturer's instruction.

### 3.2 Production of TEV-Gly5-Tn5

### Materials:

a. HEGX Buffer (20 mM HEPES-KOH at pH 7.2, 0.8 M NaCl, 1 mM EDTA, 10% glycerol, 0.2% Triton X-100)
b. 2X Tn5 Dialysis Buffer (100 mM HEPES-KOH PH 7.2, 0.2 M NaCl, 0.2 mM EDTA, 2 mM DTT, 0.2% Triton X-100, 20% Glycerol)
c. Elution buffer (10 mL HEGX with 100 mM DTT)
d. T7 Express lysY Competent E. coli (NEB C3010I)
e. IPTG (Sigma-Aldrich I1284)
f. cOmplete™, EDTA-free Protease Inhibitor Cocktail (04693132001 Roche)
g. Poly(ethyleneimine) solution (P3143 Supelco)
h. Protein concentrator (Thermo Scientific, 88502)

### Steps:

1. Transform TEV-Gly5-Tn5 plasmid to T7 Express lysY Competent E. coli (NEB C3010I), incubate overnight at 30 C.
2. Make a starter 3 ml LB-Ampicillin (100 ug/mL) culture of TEV-Gly5-Tn5 by picking a single colony from a freshly prepared streak from glycerol stock. Shake 4 hours at 37C.
3. Dilute the starter at ratio 1:100 to 300 mL LB-Ampicillin (100 ug/mL) culture. Shake at 37C until OD600˜0.6.
4. Chill the culture to 10C by cooling on ice water bath.
5. Add IPTG to 0.25 mM.
6. Shake the induced culture at 23 C for 4 hours, until the OD600˜3.0.
7. Centrifuge the culture and store the pellet at −80C.
8. Resuspend the pellet in HEGX buffer with proteinase inhibitor cocktail and sonicate the cell-buffer mixture until adequate lysis. 50% duty cycle, 30 s on 30 s off 10 cycles, 5 minutes on time.
9. Centrifuge the lysate 15000 rpm for 30 minutes at 4 C, transfer the supernatant to another 50 mL ultra-centrifugation tube with a magnetic stirrer.
10. Place the tube with supernatant in a ice-water slurry on a stirrer. Turn on mild stirring and add 2.1 ml 10% PEI dropwise. Remove the stir-bar.
11. Centrifuge the lysate at 12000 for 10 minutes at 4C.
12. Load the supernatant on a 10 mL chitin column at 0.4 mL/min in HEGX with PIC. (cap and mix overnight at 4C, wrap in parafilm)
13. Wash the column with 300 mL HEGX at 0.2 mL/min. (use more buffer to lower the contamination)
14. Add 24 mL Elution buffer to the column.
15. Drain 11 mL of the buffer then close the column for incubation at 4C for 36 to 48 hours.
16. Collect the eluent at 1 ml aliquot and pool aliquot with high protein concentration by testing with Bradford assay reagent.
17. Dialyze the pooled eluent against 2 changes of 2X Tn5 Dialysis Buffer.
18. Concentrate the dialysate with 30K MWCO protein concentrator.

### Example 4: Preparation of Covalently Linked Tn5 Transposase-Recombinant Antibody Complex

### Materials:

a. TEV-Gly5-Tn5 in 50% glycerol stock.
b. TEV-protease
c. Sortase A5
d. 1M CaCl2
e. AbFlex antibody
f. Ni-NTA Magnetic beads
g. 1M Imidazole
h. 2X Tn5 Dialysis buffer without DTT and EDTA and glycerol (100 mM HEPES-KOH PH7.2, 200 mM NaCl, 0.2% Triton X-100)
i. Protein Concentrator with 30K MWCO (Thermo 88502)

### Steps:

1. Add 150 ug of TEV-Gly5-Tn5 to a 1.5 mL lo-bind tube, top up to 100 uL with 2X Tn5 Dialysis buffer without DTT and EDTA
2. Add 1 uL TEV-protease to the lo-bind tube.
3. Incubate the tube in a thermo mixer 30 degree C., shaking 1000 rpm for 1 hour.
4. Add 1 uL 1M CaCl2 to the tube.
5. Add 0.25 uL Sortase A5 and 25 ug Antibody to the tube.
6. Incubate the tube in a thermo mixer 30 degree C., shaking 1000 rpm for 2 hours.
7. To 2 new PCR tubes, add 25 uL Ni-NTA Magnetic beads each.
8. Stand on magnetic stand to remove buffer.
9. Add 200 uL 2X Tn5 Dialysis buffer without DTT and EDTA and glycerol to resuspend the beads in each tube by light vortex
10. Stand on magnetic stand to remove buffer.
11. Repeat wash once. Discard the buffer in the first tube while keeping the second tube buffer.
12. To the 125 uL sortagging reaction mixture, add 2.5 uL 1M Imidazole
13. Add the sortagging reaction mixture to the first tube of magnetic beads.
14. Mix on a rotator at 4° C. for 30 minutes
15. Stand on magnetic stand to collect the supernatant. KEEP the supernatant!
16. Wash the beads with 10 uL 2X Tn5 Dialysis buffer without DTT and EDTA and glycerol, pool the supernatant.
17. Remove the buffer in the second tube of magnetic beads.
18. Add the supernatant to the second tube of magnetic beads.
19. Mix on a rotator at 4° C. for 30 minutes
20. Stand on magnetic stand to collect the supernatant. Keep the supernatant.
21. Wash the beads with 10 uL 2X Tn5 Dialysis buffer without DTT and EDTA and glycerol, pool the supernatant.
22. Concentrate the supernatant with Protein Concentrator with 30K MWCO.
23. Store ˜25 uL concentrate in −20C.

### Example 5: Preparation of Adapter-Loaded Tn5-Recombinant Antibody Complex

### Materials for Oligo Annealing:

a. Annealing buffer [10 mM Tris pH 8, 1 mM EDTA, 50 mM NaCl]

### Steps for Oligo Annealing:

1. Centrifuge tubes of oligo at 12000 rcf for 2 minutes to make sure all powder in the bottom of the tube.
2. Resuspend the oligo in appropriate volume of Annealing buffer to make 200 uM.
3. Mix equal volume of Tn5MErev with equal volume of Tn5ME-A/Tn5ME-B, pipette mix well.
4. Perform annealing in a PCR machine with the following program with the heated lid on:
   95 degree C. for 2 minutes;
   0.2 degree C. per minutes ramp to 4 degree C.
5. Store the annealed oligos at 20 degree C.

### Materials for Adapter Loading:

a. Concentrated Tn5-recombinant antibody complex
b. Annealed Tn5ME-A/Tn5ME-B adapters

### Steps:

1. Mix Concentrated Tn5-recombinant antibody complex with adapter in a ratio of 7:1. The adapter can be Tn5ME-A or Tn5ME-B or equal volume mix of Tn5ME-A and Tn5ME-B.
2. Incubate the mixture in thermo mixer, 23C for 30 minutes, 600 rpm.
3. Store the mixture in −20° C. until use.

### Example 6: Profiling of Co-Occurring Chromatin Features

### Materials:

a. Con-A beads (Vazyme N515)
b. 1X Binding Buffer [20 mM HEPES pH 7.9, 10 mM KCl, 1 mM CaCl2, 1 mM MnCl2]
c. Wash buffer [20 mM HEPES pH 7.5, 150 mM NaCl, 0.5 mM spermidine, 1X Roche Complete Protease Inhibitor EDTA-Free]
d. Antibody buffer [Wash buffer supplemented with 0.05% digitonin, 0.1% BSA, 2 mM EDTA]
e. Dig-300 Buffer [20 mM HEPES pH 7.5, 300 mM NaCl, 0.01% Digitonin, 0.5 mM spermidine, 1X Roche Complete Protease Inhibitor EDTA-Free]
f. adapter loaded Tn5-antibody complex
g. 1X Tagmentation Buffer [Dig-300 Buffer supplemented with 10 mM MgCl2]
h. 0.5M EDTA
i. 10% SDS
j. Proteinase K (NEB P8107)
k. Qiagen MinElute PCR purification kit
l. NEBNext HiFi PCR Mix (NEB M0541S)
m. UDI indexed primer (Illumina 20091654)

### Steps:

a. Preparation of ConA beads
   i. Per Library, Add 100 uL of 1X binding buffer to a 1.5 mL low bind tube or 5 mL tube.
   ii. Resuspend ConA beads by pipetting, transfer and mix 10 uL ConA beads to the 100 uL Binding Buffer.
   iii. Place the tube with the mixture on magnetic rack and remove supernatant.
   iv. Remove the tubes from the rack and add 100 uL Binding buffer to the beads, mix by pipetting.
   v. Place the tube with the mixture on magnetic rack and remove supernatant.
   vi. Remove the tubes from the rack and add 10 uL Binding buffer to the beads, mix by pipetting.
b. Harvest cell of interest
   i. Count cell and measure Viability.
   ii. Remove medium by centrifugation at rcf 500 for 5 minutes.
   iii. At room temperature, add 500 uL 1X Wash Buffer to resuspend the cell pellet.
   iv. Remove wash buffer by centrifugation at rcf 500 for 5 minutes.
c. Cell-ConA beads mixture
   i. Per Library, add 100 uL of Wash Buffer to the washed cell pellet, transfer mixture to a 1.5 mL low bind tube.
   ii. On a bench top vortexer, turn the dial to “5” and turn on the vortex.
   iii. Open the cap of the tube containing the cell-wash buffer mixture and press on the vortexer.
   iv. Per Library, add the 10 uL of the prepared ConA beads to the cell-wash buffer mixture slowly.
   v. Incubate the mixture at room temperature for 10 minutes.
   vi. Briefly centrifuge the tube to collect the liquid to the bottom of the tube.
   vii. Place the tube on a magnetic rack and remove supernatant.
   viii. Remove the tubes from the rack.
d. Incubation with adapter loaded Tn5-antibody complex
   i. Per Library, add 50 uL of 1X cold Antibody Buffer to resuspend the bead-cells.
   ii. Resuspend by light vortexing.
   iii. Place the tube on Ice.
   iv. Add adapter loaded Tn5-antibody complex to the cell-ConA beads mixture.
   v. Rotate the tube at 4 degree C. for overnight.
e. Washing
   i. Place the tube containing the bead-cell mixture on a magnetic rack and remove supernatant.
   ii. Add 1000 uL 1X Dig-300 buffer to the tube and invert 10 times to thoroughly mix the buffer with the beads.
   iii. Place the tube on a magnetic rack and remove supernatant.
   iv. Remove the tubes from the rack.
   v. Repeat wash once more time for a total of 3 washes.
f. Activation of tagmentation
   i. Add 100 uL of 1X Tagmentation Buffer to the tube and mix by pipetting.
   ii. Incubate at 37C for 1 hour.
g. Quenching of tagmentation
   i. Place the tube in room temperature and add 10 uL 0.5M EDTA, 3 uL 10% SDS and 2.5 uL 20 mg/ml proteinase K
   ii. Resuspend by full speed vortexing fo 2 seconds.
   iii. Briefly centrifuge the bead-cell mixture to collect the liquid to the bottom of the tube.
   iv. Incubate the tube at 55C for 1 hour.
   v. Purify the mixture with MinElute Column following Manufacturer's instruction.
   vi. Elute the DNA with 23 uL Qiagen EB
h. PCR amplification
   i. Add 2.5 uL UDI indexed primer to the 22.5 uL of purified DNA.
   ii. Add 25 uL NEBNext HiFi PCR Mix to the tube.
   iii. Run the PCR for 12-14 cycles:
      72C 5 min
      98C 30 sec
      98C 10 sec
      63C 10 sec
      72C 1 min
      8C hold
i. Purification of PCR product
   i. Equilibrate beads to room temperature for at least 15 minutes.
   ii. Add 65 uL of the beads to each PCR product. Mix by pipetting. Incubate at RT for 5 minutes.
   iii. Place the mixture on a magnetic rack.
   iv. Remove supernatant.
   v. Add 200 uL 80% ethanol. And incubate for 30 seconds. Do not remove the tube from the rack.
   vi. Place the mixture on a magnetic rack.
   vii. Remove supernatant.
   viii. Repeat wash for a total of 2 washes.
   ix. Air dry the beads.
   x. Elute the product with 22 uL ultrapure Water by incubation at room temperature for 5 minutes.

### Example 7: Performance of ReACT-seq

### 7.1 ReACT-seq Recapitulate CUT&Tag

### 7.1.1 Materials and Methods

In this example, the engineered Tn5 complex was covalently linked with either H3K4me3 antibody or H3K27me3 antibody (Proteintech, 91403, 91404). The production of engineered Tn5 complex follows procedures described in Example 3. The preparation of covalently linked Tn5 transposase-recombinant antibody complex follows example 4, where the Abflex antibody used was either H3K4me3 or H3K27me3. The loading of the adapter to the Tn5-recombinant antibody complex follows procedures described in Example 5, where an equal volume mix of annealed Tn5ME-A and Tn5ME-B was used to load the Tn5-recombinant antibody complex. ReACT-seq NGS libraries were generated following procedures described in Example 6, using the Tn5ME-A and Tn5ME-B adapter loaded Tn5-recombinant antibody complex, which links to either H3K4me3 or H3K27me3 Abflex antibody.

CUT&Tag libraries were generated using the same H3K4me3 or H3K27me3 Abflex antibody used in ReACT-seq following the protocol of Kaya-Okur et al (2019) (Kaya-Okur, H. S., Wu, S. J., Codomo, C. A. et al. CUT&Tag for efficient epigenomic profiling of small samples and single cells. Nat Commun 10, 1930 (2019)).

### 7.1.2 Results

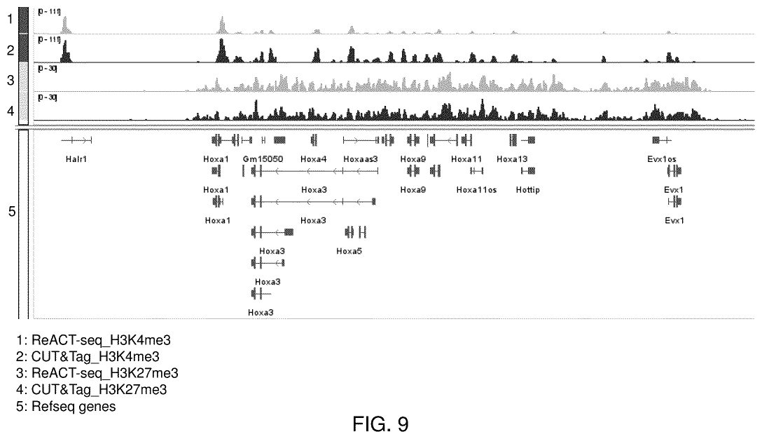

Now referring to FIG. 9, showing an alignment as coverage tracks of various reads of libraries generated with ReACT-seq protocol or method were compared with libraries generated using CUT&Tag protocol. Coverage tracks were generated by aligning the sequenced reads to mm10 reference genome using bowtie2, with the following settings: “-end-to-end-very-sensitive-no-mixed-no-discordant-I 10-X 2000”, deduplicate with picard, and convert to tracks using bedtools genomecov and bedGraphtoBigWig. Tracks showed the reads mapped to a mouse reference genome (Refseq genes). Results showed that the provided systems and methods ReACT-seq generated signal profiles similar to those of CUT&Tag for identifying a single chromatin feature.

### 7.2 ReACT-seq Identified Co-Occurrence of H3K4me3 and H3K27me3

### 7.2.1 Materials and Methods

In this example, one engineered Tn5 complex was covalently linked with H3K4me3 antibody and another engineered Tn5 complex was covalently linked with H3K27me3 antibody. The production of engineered Tn5 complex follows procedures described in Example 3. The preparation of covalently linked Tn5 transposase-recombinant antibody complex follows procedures described in Example 4, where the Abflex antibody was either H3K4me3 or H3K27me3. The loading of the adapter to the Tn5-recombinant antibody complex follows procedures described in Example 5, where annealed Tn5ME-A was used to load the Tn5 complex covalently linked with H3K4me3 antibody, and annealed Tn5ME-B was used to load the Tn5 complex covalently linked with H3K27me3 antibody. ReACT-seq NGS libraries were generated following procedures described in Example 6, using an equal volume mixture of Tn5ME-A loaded Tn5 complex covalently linked with H3K4me3 antibody and Tn5ME-B loaded Tn5 complex covalently linked with H3K27me3 antibody.

### 7.2.2 Results

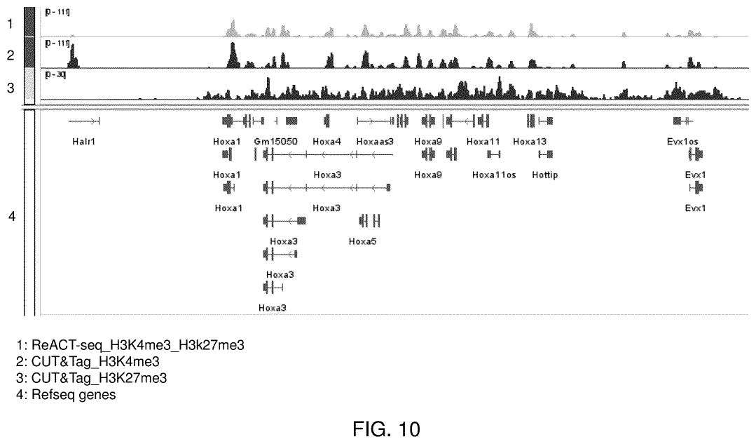

Now referring to FIG. 10, showing an alignment as coverage tracks of various reads of libraries generated with ReACT-seq protocol using Tn5 tagged with H3K4me3 and H3K27me3 antibodies, and libraries generated using CUT&Tag protocol with H3K4me3 antibody and H3K27me3 antibody individually. Coverage tracks were generated by aligning the sequenced reads to mm10 reference genome using bowtie2, with the following settings: “-end-to-end-very-sensitive-no-mixed-no-discordant-I 10-X 2000”, deduplicate with picard, and convert to tracks using bedtools genomecov and bedGraphtoBigWig. Tracks showed the reads mapped to a mouse reference genome (Refseq genes). Results showed that regions enriched with only H3K4me3, but not H3K27me3 had lower signal in the ReACT-seq generated library, compared to regions enriched with both H3K4me3 and H3K27me3. Results showed that provided systems and methods ReACT can identify co-occurring chromatin features such as H3K4me3 and H3K27me3.

7.3 ReACT-seq Identified Regions Similar to re-ChIP

### 7.3.1 Materials and Methods

In this example, the library is the same as 7.2.1. re-ChIP library data on H3K4me3 and H3K27me3 were obtained from Mas et al (2018) (Mas, G., Blanco, E., Ballaré, C. et al. Promoter bivalency favors an open chromatin architecture in embryonic stem cells. Nat Genet 50, 1452-1462 (2018)). For the re-ChIP data, peaks coordinate were converted from mm9 to mm10 using UCSC liftOver tools. For the ReACT-seq data, the peaks were called using MACS2.

### 7.3.2 Results

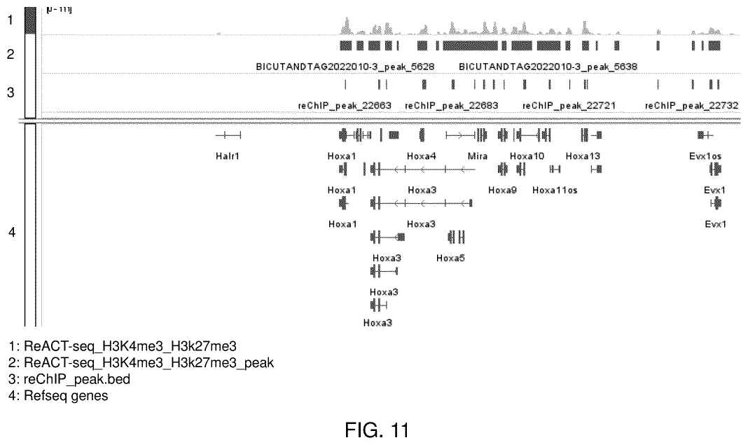

Now referring to FIG. 11, genome browser tracks showing ReACT-seq identifies regions similar to that of re-ChIP. Mouse embryonic stem cell re-ChIP data retrieved from https://doi.org/10.1038/s41588-018-0218-5. Results showed that the peaks called from ReACT-seq overlapped with peaks called from reChIP on HoxA gene cluster. Results showed that provided systems and methods ReACT can identify regions or co-occurring chromatin features similar to that of re-ChIP.

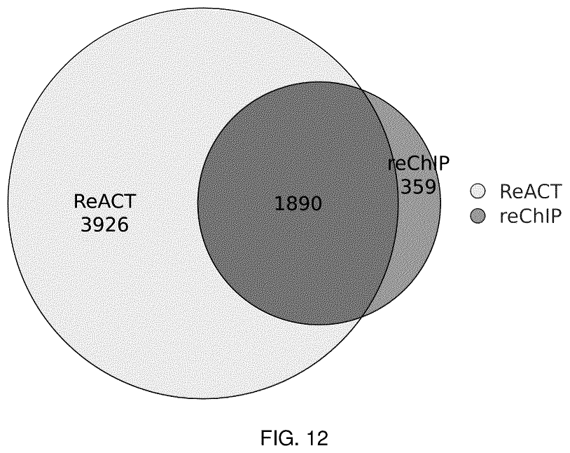

Now referring to FIG. 12, the venn diagram showed genome wide overlapping of peaks called from ReACT-seq and reChIP. Results showed that ReACT identified 5816 peaks while reChIP identified 2249 peaks. Results showed that ReACT-seq identified 1890 out of 2249 peaks from reChIP, and more peaks can be identified by ReACT-seq, indicating the outstanding performance of the provided systems and methods ReACT-seq.

The exemplary embodiments of the present invention are thus fully described. Although the description referred to particular embodiments, it will be clear to one skilled in the art that the present invention may be practiced with variation of these specific details. Hence this invention should not be construed as limited to the embodiments set forth herein.

For examples, in some examples, two transposase complexes each with a single transposase and optionally a different adapter are provided, but in other examples, the complex may comprise one or more transposase molecules and one or more adapters.

For example, a complex comprising at least two transposases, at least two of the transposases are attached to the chromatin or oligonucleotide. Higher order complexes such as 3, 4, 5, 6, 7, 8 or more transposases, or mixtures of different numbers of complexes are also possible.

For example, in complexes containing more than two transposases, not all transposases need to be bound by chromatin or oligonucleotides. Instead, it is sufficient for two of the transposases to be joined, although additional chromatin or oligonucleotides may be joined.

What is claimed is:

1. An engineered Tn5 transposase complex, comprising:
at least one Tn5 transposase, comprising a poly-glycine sequence; and
at least one antibody that is covalently linked to the at least one Tn5 transposase by a sortase,
wherein the at least one antibody is specific to a target site of a chromatin sequence.

2. The engineered Tn5 transposase complex of claim 1, further comprises at least one adapter.

3. The engineered Tn5 transposase complex of claim 2, wherein the at least one adaptor comprises at least one barcode sequence.

4. A plurality of engineered Tn5 transposase complexes as claimed in claim 1, wherein each complex comprises a different antibody that is specific to a different target site of the chromatin sequence.

5. The plurality of complex of claim 4, wherein each complex further comprises at least one different adapter comprising at least one different barcode sequence.

6. A system of profiling co-occurring chromatin features, comprising a plurality of engineered Tn5 transposase complexes as claimed in claim 4, wherein each complex comprises a different antibody comprising a different specific binding site for tagging a different specific sequence on the at least one target chromatin.

7. The system of claim 5, wherein each complex further comprises at least one different adaptor comprising at least one different barcode sequence.

8. The system of claim 6, comprising:
a first engineered Tn5 transposase complex as claimed in any one of the preceding claims, wherein the first complex comprises a first antibody comprising a first specific binding site for tagging a first specific sequence on a target chromatin; and
a second engineered Tn5 transposase complex as claimed in any one of the preceding claims, wherein the second complex comprises a second antibody comprising a second specific binding site for tagging a second specific sequence on the target chromatin,
wherein the first engineered Tn5 transposase complex further comprises at least one first adapter, and the second engineered Tn5 transposase complex further comprises at least one second adapter, and
wherein the first adapter comprises a first barcode sequence and the second adapter comprises a second barcode sequence that is different from the first barcode sequence.

9. A method of profiling co-occurring chromatin features using a plurality of complexes as claimed in claim 1 or the system of the claim 6, comprising the steps of:
(i) reacting the plurality of complexes with a sample such that the plurality of complexes bind to the any target site of the chromatin sequence of the sample to obtain a chromatin fragment,
(ii) performing tagmentation on the chromatin fragment in situ; and
(iii) preparing sequencing library for sequencing.

10. The method of claim 9, wherein each complex further comprises at least one different adaptor.

11. The method of claim 10, wherein the at least one different adaptor comprises at least one different barcode sequence.

12. The method of claim 9, prior to the step (iii), further comprises the steps of:
purifying the chromatin fragment and adding sequencing platform specific adapter at each end of the chromatin fragment by PCR.

13. The method of claim 9, wherein at least one or more of the steps are performed in a microfluidic device.

14. A method of preparing recombinant Tn5 transposase complex, comprising the steps of:
(a) preparing at least one Tn5 transposase comprising poly-glycine sequence; and
(b) covalently linking the at least one Tn5 transposase comprising poly-glycine sequence with at least one antibody comprising a LPXTG-motif tag by a sortase, wherein the at least one antibody that is specific to a target site of a chromatin sequence.

15. The method of claim 14, wherein the step (a) further comprises the steps of:
preparing the at least one Tn5 transposase comprising poly-glycine sequence by reacting TEV-Gly5-Tn5 with a TEV-protease.

16. The method of claim 15, the TEV-Gly5-Tn5 is prepared by replacing 3XFlag-pA portion of a 3XFlag-pA-Tn5-FI plasmid by PCR.

17. The method of claim 15, further comprising a step of: prior to the (b), purifying the recombinant Tn5 transposase complex.

18. The method of claim 15, further comprising a step of: reacting the recombinant Tn5 transposase complex with at least one adapters to form adaptor loaded Tn5 transposase complex.

19. The method of claim 18, wherein the at least one adaptor comprises at least one barcode sequence.


SEQUENCE LISTING (2 sequences)

SEQ ID NO: 1 - DNA, 34 bp, synthetic construct
  source 1..34: mol_type = other DNA
ggtggtggcggcggtgatgacgataaagaattcg

SEQ ID NO: 2 - DNA, 36 bp, synthetic construct
  source 1..36: mol_type = other DNA
ttggaaatacaggttctccatggtatatctccttct